SEQUENCE LISTING (2 sequences)

SEQ ID NO: 1 - DNA, 12 bp, Homo sapiens
ctgggtcataga

SEQ ID NO: 2 - DNA, 11 bp, Artificial Sequence
  Description of Artificial Sequence Synthetic oligonucleotide
tctatgaccca

(19) United States
(12) Patent Application Publication
(10) Pub. No.: US 2021/0062265 A1
(43) Pub. Date: Mar. 4, 2021

(54) COMPOSITIONS AND METHODS FOR IDENTIFYING A SINGLE-NUCLEOTIDE VARIANT

(71) Applicants: Children's Medical Center Corporation, Boston, MA (US); President and Fellows of Harvard College, Cambridge, MA (US)

(72) Inventors: Michael A. Lodato, Boston, MA (US); Craig L. Bohrson, Cambridge, MA (US); Michael E. Coulter, Cambridge, MA (US); Peter J. Park, Cambridge, MA (US); Christopher A. Walsh, Boston, MA (US); Rachel E. Rodin, Boston, MA (US); Alison Barton, Cambridge, MA (US); Minseok Kwon, Cambridge, MA (US)

(73) Assignees: Children's Medical Center Corporation, Boston, MA (US); President and Fellows of Harvard College, Cambridge, MA (US)

(21) Appl. No.: 16/984,885

(22) Filed: Aug. 4, 2020

Related U.S. Application Data
(63) Continuation of application No. PCT/US2019/017292, filed on Feb. 8, 2019.
(60) Provisional application No. 62/628,575, filed on Feb. 9, 2018.

(51) Int. Cl. C12Q 1/6883; C12N 15/10

(52) U.S. Cl. CPC C12Q 1/6883; C12Q 2600/118; C12Q 2600/156; C12N 15/1065

(57) ABSTRACT

Provided are compositions and methods of identifying a single-nucleotide variant (sSNV) in a single cell which involve detecting a variant nucleotide on forward and reverse strands of genomic DNA, wherein the presence of the variant nucleotide on the forward and reverse strands identifies a double-stranded mutation that is a single-nucleotide variant.

### CROSS-REFERENCE TO RELATED APPLICATION

This application is a U.S. national stage application under 35 U.S.C. 111(a) that is a continuation of and claims the benefit of and priority to PCT Application No.: PCT/US19/17292, filed Feb. 8, 2019, which claims the benefit of and priority to U.S. Provisional Application No. 62/628,575, filed Feb. 9, 2018, the entire contents of each of which are incorporated herein by reference.

### STATEMENT OF RIGHTS TO INVENTIONS MADE UNDER FEDERALLY SPONSORED RESEARCH

This invention was made with government support under Grant Nos. K99 AG054749 01, T32GM007753, F30 MH102909, 1S10RR028832-01, T32HG002295, U01MH106883, P50MH106933, R01 NS032457, and U01 MH106883 R01 NS032457, R01 NS079277 and U01 MH106883 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 29, 2020, is named 167705_014802US_SL.txt and is 800 bytes in size.

### BACKGROUND OF THE INVENTION

Aging in humans brings increased incidence of nearly all diseases, including neurodegeneration. Markers of DNA damage increase in the brain with age, and genetic progeroid diseases, such as Cockayne syndrome (CS) and Xeroderma pigmentosum (XP), both caused by defects in DNA damage repair (DDR), are associated with neurodegeneration and premature aging. Mouse models of aging, CS, and XP have shown inconsistent relationships between these conditions and the accumulation of permanent somatic mutations in brain and non-brain tissue. While analysis of human bulk brain DNA, comprising multiple proliferative and non-proliferative cell types, revealed an accumulation of mutations during aging in the human brain, it is not known whether permanent somatic mutations accumulate with age in mature neurons of the human brain. Accordingly, a need exists for improved compositions and methods for examining whether permanent somatic mutations accumulate during aging in the human brain.

### SUMMARY OF THE INVENTION

As described below, the present disclosure features compositions and methods of identifying a single-nucleotide variant (SNV) in a single cell by detecting a variant nucleotide on forward and reverse strands of genomic DNA, wherein the presence of the variant nucleotide on the forward and reverse strands identifies a double-stranded mutation that is a single-nucleotide variant. Methods of the invention are useful for determining the genomic age of a subject, measuring the rate of accumulation of genome-wide somatic single-nucleotide variants (sSNVs), and/or measuring somatic mutation burden.

In one aspect, the invention features a method of identifying a single-nucleotide variant (SNV) in a single cell, the method involving (a) purifying genomic DNA from a single cell; (b) amplifying the genomic DNA; (c) sequencing the amplified genomic DNA; (d) detecting a variant nucleotide on forward and reverse strands, wherein the presence of the variant nucleotide on the forward and reverse strands identifies a double-stranded mutation that is a single-nucleotide variant. In one embodiment, the single-nucleotide variant is a somatic mutation. In another embodiment, detecting the variant nucleotide is in proximity to a germline variant. In another embodiment, the cell is a neuron, cardiac cell, muscle cell, or skin cell. In another embodiment, the purifying step comprises alkaline lysis on ice. In another embodiment, the purifying step comprises isolating the nucleus from the single cell. In another embodiment, the method further involves eliminating from a sequence read a variant nucleotide that is not present on forward and reverse strands, wherein the absence of the variant nucleotide on a forward or reverse strand identifies an error in genomic sequencing. In another embodiment, the error is a DNA lesion. In another embodiment, the DNA lesion is biologically induced pre-mortem, biologically induced post-mortem, or generated during DNA purification, nuclear isolation, cell lysis, DNA amplification, DNA library preparation, or DNA sequencing. In another embodiment, the error is a chemically induced DNA lesion generated by cell lysis conditions. In another embodiment, the error is generated by DNA amplification. In another embodiment, sequence data for forward and reverse strands is obtained.

In another aspect, the invention provides a method of determining the genomic age of a subject, the method involving (a) purifying genomic DNA from a single cell obtained from the subject; (b) amplifying the genomic DNA; (c) sequencing the amplified genomic DNA; (d) measuring the number of somatic variant nucleotides on forward and reverse strands, wherein the presence of the somatic variant nucleotide on the forward and reverse strands identifies a double-stranded mutation that is a somatic single-nucleotide variant; and (e) determining a genomic age from the number of somatic variant nucleotides relative to a reference, wherein increased somatic variant nucleotides relative to a reference indicates advanced genomic age. In one embodiment, the method comprises measuring the number of somatic variant nucleotides in at least 2, 3, 4, 5 or more cells. In another embodiment, the somatic variant nucleotide is in proximity to a germline variant.

In another aspect, the invention features a method of measuring the rate of accumulation of genome-wide somatic single-nucleotide variants (SNVs), the method comprising:

identifying double-stranded mutations in a genomic sequence, wherein the double-stranded mutations comprise a variant nucleotide on forward and reverse strands; and

performing linkage analysis to obtain a frequency of observed double-stranded mutations adjusted for the number of regions linked to a germline heterozygous variant, thereby measuring the rate of accumulation of genome-wide somatic SNVs. In one embodiment, the genomic sequence is obtained from a biological sample of a subject. In another embodiment, the biological sample is a single cell or single nucleus. In another embodiment, the cell is a neuron, cardiac cell, muscle cell, or skin cell. In another embodiment, the nucleus is obtained from a neuron, cardiac cell, muscle cell, or skin cell.

In another aspect, the invention features a method of measuring the somatic mutation burden of a subject, the method comprising:

identifying double-stranded mutations in a genomic sequence from a biological sample from the subject, wherein the double-stranded mutations comprise a variant nucleotide on forward and reverse strands; and

performing linkage analysis to obtain a frequency of observed double-stranded mutations adjusted for the number of regions linked to a germline heterozygous variant, thereby measuring the somatic mutation burden in the subject. In one embodiment, increased somatic mutation burden in the subject indicates advanced age and/or increased DNA damage. In another embodiment, the rate of somatic mutation burden of greater than about 20 SNVs per year in the prefrontal cortex (PFC) and/or greater than about 40 SNVs per year in the hippocampal dentate gyrus (DG) is indicative of neuronal degeneration in the subject. In another embodiment, the neuronal degeneration is associated with Cockayne syndrome (CS) or Xeroderma pigmentosum (XP).

In various embodiments of the above aspects, the cell is a neuron, cardiac cell, muscle cell, or skin cell. In various embodiments of the above aspects, the subject has or is identified as having a progeroid disease (e.g., Cockayne syndrome (CS) or Xeroderma pigmentosum (XP)). In various embodiments of the above aspects, =the purifying step comprises alkaline lysis on ice. In various embodiments of the above aspects, the purifying step comprises isolating the nucleus from the single cell. In various embodiments of the above aspects, the method further involves measuring the rate of accumulation of genome-wide somatic SNVs. In various embodiments of the above aspects, the cell or cells is obtained from a biological sample of a subject.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant a change (increase or decrease) in the expression levels or activity of a gene or polypeptide as detected by standard art known methods such as those described herein. As used herein, an alteration includes a 10% change in expression or activity levels, preferably a 25% change, more preferably a 40% change, and most preferably a 50% or greater change in expression levels.

In this disclosure, “comprises,” “comprising,” “containing,” and “having” and the like can have the meaning ascribed to them in U.S. Patent law, and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “disease” is meant any condition or disorder that damages or interferes with the normal function of a cell, tissue, or organ. Examples of diseases include, without limitation, neurodegeneration and genetic progeroid diseases, such as Cockayne syndrome (CS) and Xeroderma pigmentosum (XP).

The terms “isolated,” “purified,” or “biologically pure” refer to material that is free, to varying degrees, from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Primer set” means a set of oligonucleotides that may be used for DNA amplification, for example, PCR. A primer set would consist of at least 2, 4, 6, 8, 10, 12, 14, 16, 18, 20, 30, 40, 50, 60, 80, 100, 200, 250, 300, 400, 500, 600, or more primers.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

The term “variant” is used herein to refer to a change or alteration to a reference genomic DNA at a particular locus (e.g., in human), including, but not limited to, nucleotide base substitutions, deletions, insertions in the coding and non-coding regions.

The term “single nucleotide variant” (SNV) refers to a single nucleotide alteration at a particular site. SNVs occur without any limitations of frequency and may arise in somatic cells, e.g., a “somatic single-nucleotide variant (sSNV).” In various embodiments, the sSNV is identified by the presence of a complementary nucleotide (G-C; A-T) on the opposite strand.

The term “single nucleotide polymorphism” (SNP) refers to a single nucleotide alteration at a particular site that occurs in at least about 1% of the general population of a species. In the human genome, single nucleotide polymorphisms occur about once in every 300 nucleotide base pairs. SNPs may or may not be located within genes and may or may not affect gene expression or protein function.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1B depict detection of somatic single-nucleotide variants (sSNVs) across individuals and brain regions using single-neuron whole-genome sequencing (WGS) and linkage-based analysis. FIG. 1A shows an experimental outline. FIG. 1B is a schematic showing linkage-based mutation calling. A somatic mutation (solid arrow labeled “Somatic mutation (e.g., age-related)”) may occur on one allele (Allele 1) of a locus (Locus 1), potentially in close proximity to a germline heterozygous site (dashed arrows), while other loci, such as Locus 2, remain unmutated. Later amplification errors could create a mismatch (solid arrow labeled “Amplification error”) on one strand of one allele of Locus 2 near a germline variant. For Locus 1, any WGS read that covers both sites and contains the germline variant should also contain the somatic variant, thus these variants are perfectly linked. In contrast, at Locus 2, some, but not all, reads that cover the relevant germline variant will contain the somatic “variant” candidate, generating two classes of reads, some with the somatic variant on that allele, some without. Only perfectly linked sSNV candidates were considered in this study (see bottom left of FIG. 1B).

FIGS. 2A-2B are graphs showing that linkage analysis identifies high-confidence somatic mutations. To determine the quality of the sSNVs, single cell data was identified and their alternate allele fractions were compared to those of germline variants. FIG. 2A is a graph that shows alternate allele fraction distributions for high-confidence (1000 Genomes) heterozygous germline variants close enough to other germline heterozygous sites to be subject to read-backed linkage analysis. The variants from all samples were pooled and only variants meeting the same filtering criteria as the somatic set are shown. FIG. 2B shows the same plot for candidate somatic mutations. The distributions are comparable, indicating that the somatic mutation calling method using linkage analysis resulted in variants that are likely to be true positives.

FIGS. 3A-3I are graphs showing the correlation of various quality control metrics with estimated single-neuron sSNV counts. Positive correlation coefficients were lower than those observed between sSNV counts and age. Some metrics negatively correlated with the estimated sSNV count. * denotes p<0.05, one-way Anova with Sidak's correction for multiple testing. Circles, triangles, and diamonds denote normal prefrontal cortex (PFC) neurons, normal dentate gyrus (DG) neurons, and progeroid PFC neurons, respectively. Neurons are color-coded by individual based on the key in FIG. 1.

FIGS. 4A-4E are graphs showing that sSNVs increase with age in the PFC and DG, and are elevated in Cockayne syndrome (CS) and Xeroderma pigmentosum (XP). FIG. 4A is a graph showing that sSNV counts plotted against age for neurons derived from PFC (circles, left) and DG (triangles, right), with linear regression lines. There is a strong correlation with age in both cases, with the rate of accumulation being nearly two-fold higher in the DG than in the PFC. FIG. 4B is a graph showing the comparison of sSNV counts in matched PFC and DG within brains. FIG. 4C is a graph showing CS and XP patient neurons displaying elevated sSNV counts. FIG. 4D and FIG. 4E are graphs showing a fraction of sSNVs comprised of C>T (FIG. 4D) and T>C (FIG. 4E). *, **, and *** denote p≤0.05, 0.001, and 0.0001, respectively, using 2-way ANOVA with Sidak's correction.

FIGS. 5A-5C are graphs showing group-level comparisons of single-neuron sSNV counts between PFC neurons (FIG. 5A), DG neurons (FIG. 5B), and between neurons from the PFC compared to the neurons from the DG across age groups (FIG. 5C). * denotes p<0.05, and ** denotes p<0.01.

FIGS. 6A-6C are graphs showing the type of substitution observed. sSNV types were observed in normal PFC neurons (FIG. 6A), normal DG neurons (FIG. 6B), and CS and XP neurons (FIG. 6C). Mean per group is shown, and error bars denote SD.

FIG. 7 depicts four graphs showing the correlation of C>T and T>C mutations with age in the PFC or DG. Mean and SD were plotted for each case. Pearson R2 displayed on each plot denotes correlation between the fraction of the indicated sSNV type and age.

FIG. 8 is a graph showing enrichment of sSNVs in introns (left bar in each set (PFC, DG, CS, and XP)), exons (middle bar in each set), and intergenic regions (right bar in each set). * denotes p<0.05 for enrichment of sSNVs in the indicated feature, + denotes p<0.05 for depletion of sSNVs in the indicated genomic feature, ++ denotes p<0.001 depletion of sSNVs in the indicated genomic feature. Error bars denote a 95% confidence interval.

FIG. 9 depicts nine graphs showing strand bias in transcribed regions in all groups (e.g., infant PFC, Adolescent (Adol.) PFC, Adult PFC, Aged PFC, Adol. DG, Adult DG, Aged DG, CS, and XP). Mean and SD for each group is plotted. ** denotes p<0.01, *** denotes p≤0.001, and **** denotes p<0.0001, Two-Way ANOVA with Sidak's correction for multiple testing.

FIGS. 10A-10B show a graph and a schematic illustration depicting gene ontology and identified exonic mutations. FIG. 10A is a graph showing selected gene ontology terms enriched for sSNVs in single neurons across all groups. FIG. 10B is a schematic illustration showing gene models of heterozygous somatic mutations causing predicted deleterious mutations. In several neurons, deleterious somatic mutations were found in genes associated with dominant neurological diseases, including a predicted deleterious missense mutation in the glutamate receptor, GRIN2D, in a neuron from XP Case 5416, which has been implicated in dominant early infantile epileptic encephalopathy (M. Lynch, Proc Natl Acad Sci USA 107, 961-968 (2010)), and a nonsense mutation in RAI-1, which is a haploinsufficient circadian rhythm protein mutated in the intellectual disability disease, Smith-Magenis syndrome (K. Baynton, R. P. Fuchs, Trends Biochem Sci 25, 74-79 (2000)), in a DG neuron from Case 5532.

FIGS. 11A-11C show three graphs modeling the rate of accumulation of biallelic gene knockouts due to somatic mutations in single human neurons. FIG. 11A is a graph showing the estimate of probability of a neuronal genome containing at least one biallelic gene knockout per deleterious exonic mutation. FIG. 11B is a graph showing data-derived approximation for the accumulation of neurons with gene knockouts based on single-neuron sSNV counts. The estimated percent of neurons with at least one gene knockout (KO neurons) in a population is plotted against the total mutational load, and would theoretically saturate the genome between 250,000-300,000 sSNVs. Inset, is the expected percent of KO neurons within the range of sSNV counts measured in this study. FIG. 11C is a graph showing the general estimate of the number of KO neurons by tissue, plotted by age. Light gray band through normal PFC neurons indicates a normal increase of KO neurons with age. Nearly all CS and XP diseased neurons have SNV rates corresponding to greater than ˜0.01% KO neurons. KO neuron rates in normal infant, adolescent and adult PFC are generally below this level, while normal aged samples are also predicted to have greater than 0.01% KO neurons, suggesting that aged PFC samples may be at or near a “KO neuron burden” associated with neurological dysfunction. Moreover, the elevated levels of somatic SNVs in normal DG neurons predict much higher estimated rates of KO neurons relative to PFC, so that the exponential relationship would sharpen potential regional variation in the onset of neuronal dysfunction with age.

FIGS. 12A-121 are graphs showing that signature analysis reveals mutational processes during aging, across brain regions, and in disease. FIG. 12A are graphs showing three mutational signatures identified by matrix factorization (each substitution is classified by its trinucleotide context). FIG. 12B is a graph showing the number of variants from Signatures A, B, and C in each of the 161 neurons in the dataset. FIG. 12C, FIG. 12D, and FIG. 12E are graphs showing that Signature A strongly correlates with age, regardless of disease status or brain region, while Signatures B and C do not. FIG. 12F is a graph showing signature B is enriched in DG relative to PFC neurons. FIG. 12G is a graph showing signature B increased with age in DG neurons, but not in matched PFC neurons, revealing a DG-specific aging signature. Solid shapes represent regional means, and transparent shapes represent individual neurons. FIG. 12H and FIG. 12I are graphs showing the comparison of age-corrected estimate of sSNVs per signature in CS and XP compared to PFC controls revealed enrichment in Signature C in both CS and XP. * and ** denote p≤0.05 and p≤0.001, respectively, mixed linear model.

FIGS. 13A-13C are graphs and images showing the non-negative matrix factorization signatures, stability, and clustering. FIG. 13A is a graph showing a detailed view of identified mutational signatures. FIG. 13B is a graph showing the non-negative matrix factorization signature stability compared to average Frobenius reconstruction error indicates three signatures maximizes the number of identified signatures while minimizing error. FIG. 13C is an image of a correlation-matrix-based unsupervised hierarchical clustering of single-neuron-derived signatures (Signature A, B, and C) with published COSMIC signatures (Signature 1-30) (L. B. Alexandrov et al., Nat Genet 47, 1402-1407 (2015)).

FIGS. 14A-14H are graphs showing mutational signature analysis excluding C>T mutations. To investigate the possibility that C>T sSNVs may represent post-mortem artifacts, all C>T SNVs were removed from the results and the non-negative matrix factorization (NMF) was re-calculated to derive mutational signatures. Three mutational signatures A, B, and C were once again derived from the data set, and it was found that the relationships between Signatures A, B, and C and aging, brain region, and progeroid disease, respectively, were generally consistent with the findings when all were considered variant. FIG. 14A is a graph showing non-negative matrix factorization derived three signatures from the dataset when C>T mutations were excluded. FIG. 14B, FIG. 14C, and FIG. 14D are graphs showing that Signature A excluding C>T increases with age, while Signatures B and C do not. FIG. 14E and FIG. 14F are graphs that showing Signature B is enriched in DG relative to matched PFC, but does not increase with age in either brain region. FIG. 14G and FIG. 14H are graphs showing Signature B is increased in both CS and XP relative to age-corrected PFC, while Signature C is non-significantly increased in CS and XP. * denotes p<0.05. The data presented in this figure suggested that bona fide C>T transitions were drivers of somatic mosaicism in human neurons, and that the signatures identified here are robust to the loss of signal from a major component of the data.

FIGS. 15A-15C are graphs showing that patterns of somatic mutation differ across tissues. FIG. 15A is a graph showing the comparison of somatic SNV counts per cell in adolescent cortical neurons (ages 15-19) and muscle cells from two individuals (ages 17 and 20), demonstrating that muscle nuclei have more mutations. FIG. 15B is a graph showing the molecular signature analysis of mutations discovered across a large dataset of neurons and muscle cells identified four signatures. FIG. 15C is a graph showing Signature 4 was significantly different between muscle and brain cells, with muscle cells having more Signature 4 mutations than brain cells.

FIG. 16 is a table showing the case information and number of neurons analyzed in the experiments of this disclosure.

FIG. 17 is a table showing germline mutations in the ERCC6 gene (also known as CSB), XPA gene, and XPD gene detected in bulk WGS data from each CS and XP case. FIG. 17 discloses SEQ ID NOs: 1 and 2, respectively, in order of appearance.

### DETAILED DESCRIPTION OF THE INVENTION

The present disclosure features compositions and methods for determining the genomic age of a subject, measuring the rate of accumulation of genome-wide somatic single-nucleotide variants (SNVs), and/or measuring somatic mutation burden. The methods involve identifying a single-nucleotide variant (sSNV) in a single cell by detecting a variant nucleotide on forward and reverse strands of genomic DNA, wherein the presence of the variant nucleotide on the forward and reverse strands identifies a double-stranded mutation that is a single-nucleotide variant.

The invention is based, at least in part, on the discovery that somatic single-nucleotide variants accumulate in human neurons in aging with regional specificity and in progeroid diseases (e.g., Cockayne syndrome (CS) and Xeroderma pigmentosum (XP)). It has long been hypothesized that aging and neurodegeneration are associated with somatic mutation in neurons. However, methodological hurdles have prevented testing this hypothesis directly. In the present disclosure, it was quantitatively examined whether aging or disorders of defective DNA damage repair (DDR) result in more somatic mutations in single postmitotic human neurons. As described in the experiments below, single-cell whole-genome sequencing was used to perform genome-wide somatic single-nucleotide variant (sSNV) identification on DNA from 161 single neurons from the prefrontal cortex and hippocampus of fifteen normal individuals (aged 4 months to 82 years) as well as nine individuals affected by early-onset neurodegeneration due to genetic disorders of DNA repair (Cockayne syndrome and Xeroderma pigmentosum). sSNVs increased approximately linearly with age in both areas (with a higher rate in the hippocampus) and were more abundant in individuals having CS or XP early-onset neurodegeneration. Cells from newborns have between 300-900 sSNVs per genome, aged people (over age 75) have about 2500 sSNVs per genome. Aged people within dentate gyrus have 4000 sSNVs per genome. The accumulation of somatic mutations with age—which is termed herein as “genosenium”—shows age-related, region-related, and disease-related molecular signatures, and may be important in other human age-associated conditions.

Single Cell Whole Genome Sequencing

Whole genome sequencing (also known as “WGS”) is a process that determines the DNA sequence of an organism's genome. In various embodiments, the genome of a single cell is sequenced, including from a nucleus isolated from the cell. Methods of isolating single cells and nuclei of cells are known in the art. For single cell WGS, whole genome amplification is used to construct a library. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Sequencing of library fragments can be determined by any known method for DNA sequencing. However, high throughput sequencing methods are generally preferred. In one embodiment, the sequencing of a DNA fragment is carried out using commercially available sequencing technology, e.g., SBS (sequencing by synthesis) by Illumina. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD® technology from Applied Biosystems. Any appropriate sequencing technology may be chosen for sequencing.

All sequencing libraries contain finite pools of distinct DNA fragments. In a sequencing experiment only some of these fragments are sampled. As used herein, the term “coverage” refers to the percentage of genome covered by reads. Coverage also refers to, in shotgun sequencing, the average number of reads representing a given nucleotide in the reconstructed sequence. Biases in sample preparation, sequencing, and genomic alignment and assembly can result in regions of the genome that lack coverage (that is, gaps) and in regions with much higher coverage than theoretically expected. The term depth may also be used to describe how much of the complexity in a sequencing library has been sampled.

### Kits

The invention provides kits for determining the genomic age of a subject, measuring the rate of accumulation of genome-wide somatic single-nucleotide variants (SNVs), and/or measuring somatic mutation burden. In particular embodiments, kits include one or more reagents for single cell (e.g., neuron) isolation, whole genome amplification (e.g., primers), and/or whole genome sequencing. In some embodiments, the kit comprises a sterile container containing a reagent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. If desired, the kit is provided together with instructions for identifying somatic single nucleotide variants. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology”; “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction” (Mullis, 1994); and “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Aging and Neurodegeneration are Associated with Increased Mutations in Single Human Neurons

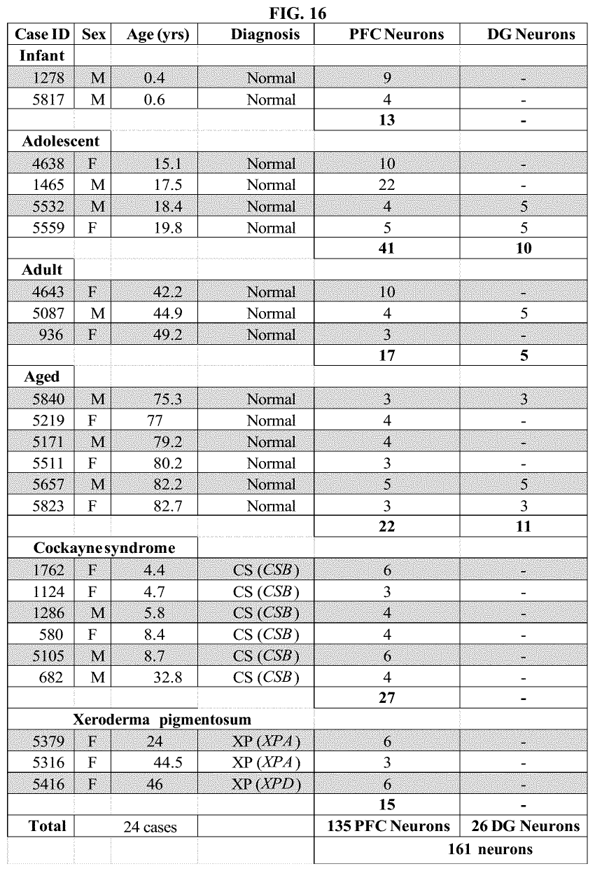

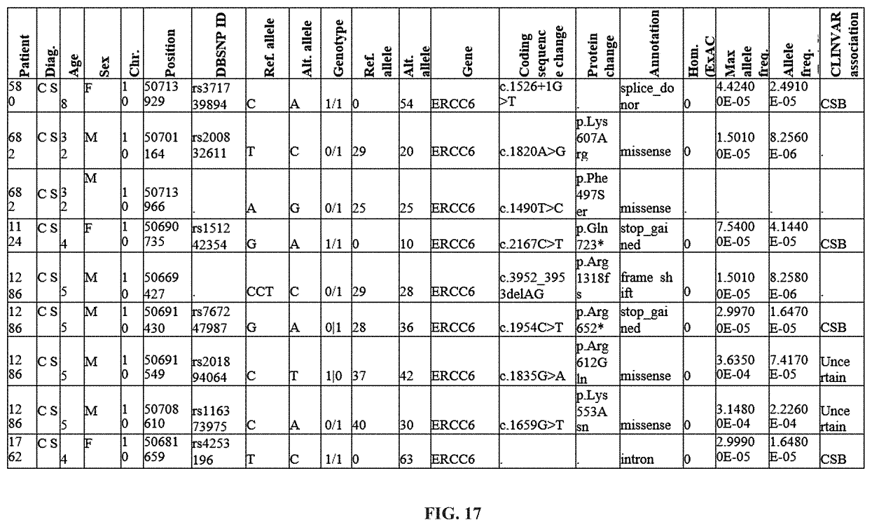

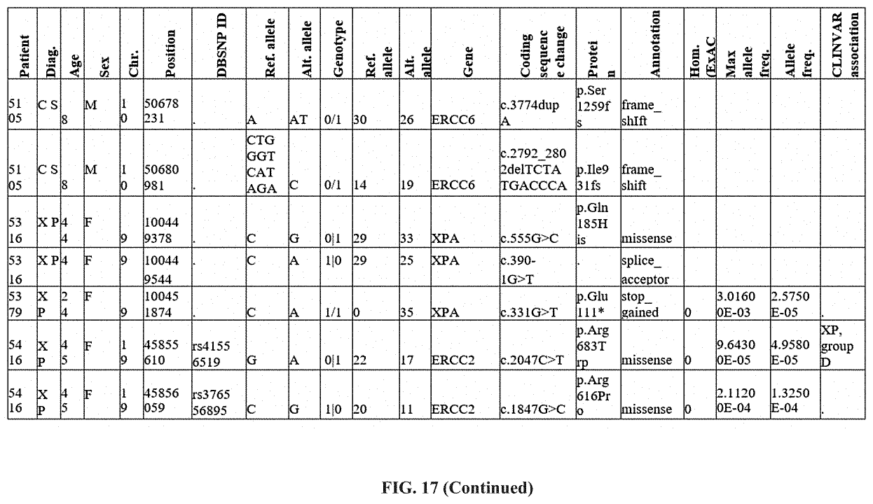

Somatic mutations that occur in postmitotic neurons are unique to each cell, and thus can only be comprehensively assayed by comparing the genomes of single cells (J. P. Dumanski et al., Methods Mol Biol 838, 249-272 (2012)). Therefore, human neurons were analyzed by single-cell whole-genome sequencing (WGS). Alterations of the prefrontal cortex (PFC) have been linked to age-related cognitive decline and neurodegenerative disease (S. J. van Veluw et al., Brain Struct Funct, (2012)). Ninety-three (93) neurons were analyzed from the PFC of 15 neurologically normal individuals from ages 4 months to 82 years (FIG. 16). Twenty-six (26) neurons from the hippocampal dentate gyrus (DG) of 6 of these individuals were further examined. The DG is a focal point for other age-related degenerative conditions, such as Alzheimer's disease. Moreover, the DG is one of the few parts of the brain that appears to undergo neurogenesis after birth (P. S. Eriksson et al., Nat Med 4, 1313-1317 (1998)), which might create regional differences in number and type of somatic mutations. Finally, to test whether defective DNA damage repair (DDR) in early-onset neurodegeneration is associated with increased somatic mutations, 42 PFC neurons from 9 individuals diagnosed with Cockayne syndrome (CS) or Xeroderma pigmentosum (XP) were analyzed (FIG. 16 and FIG. 17).

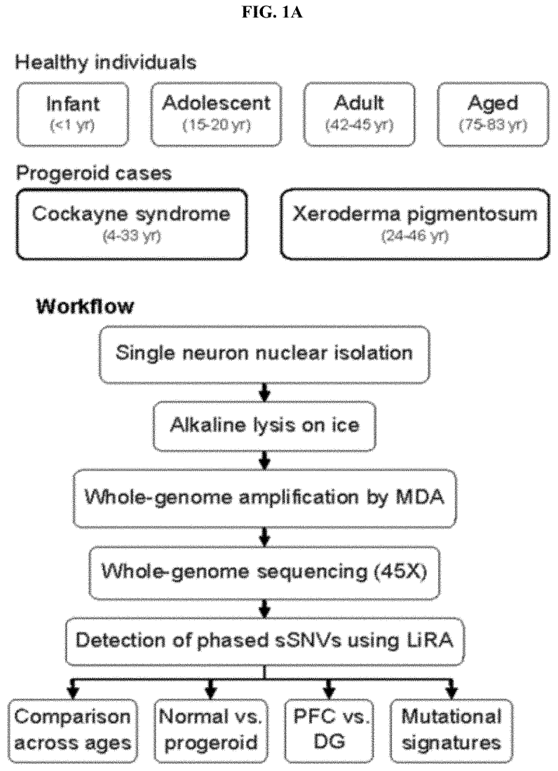

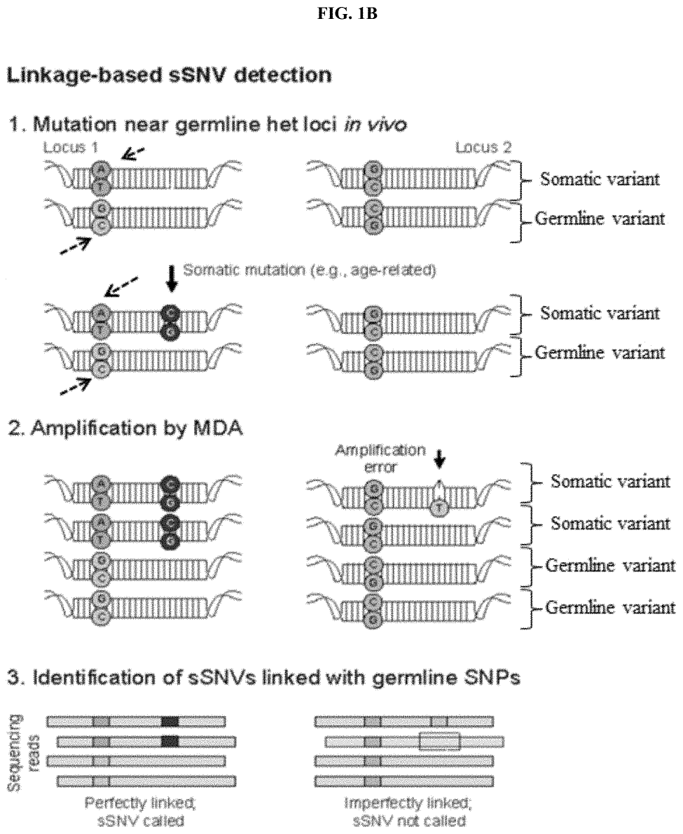

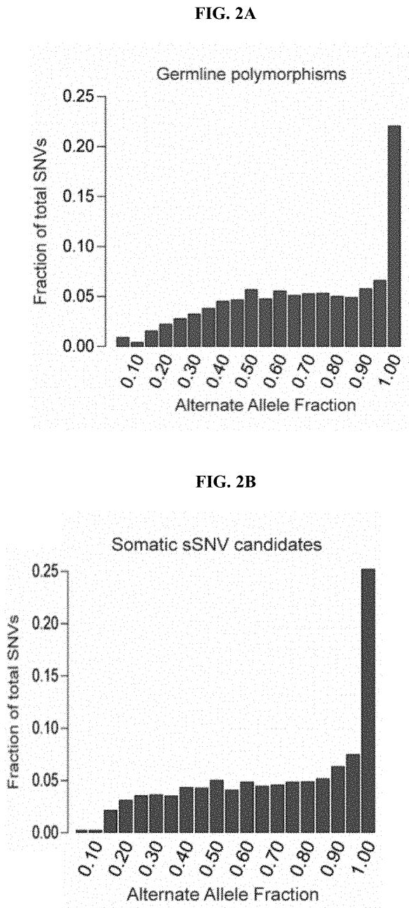

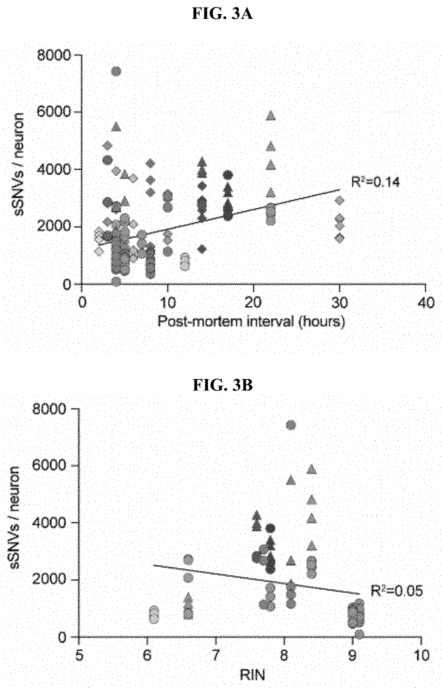

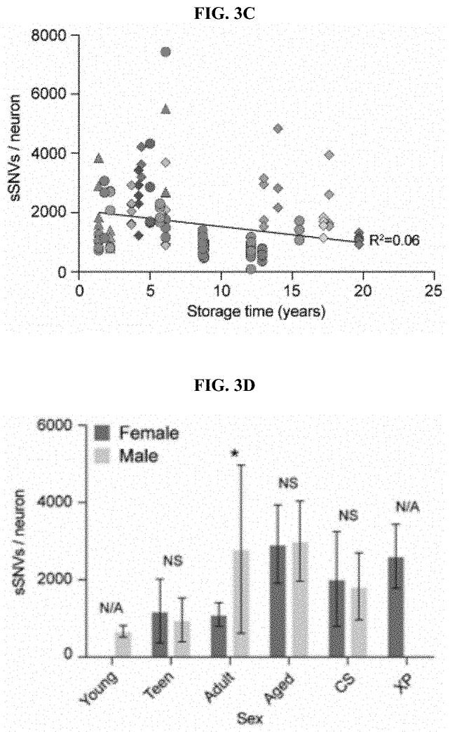

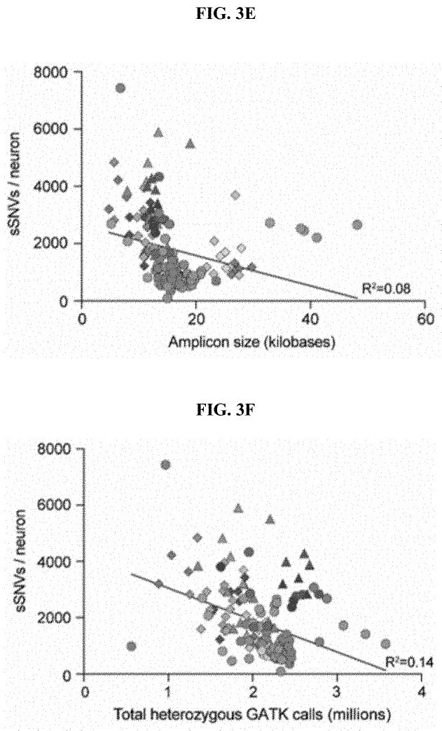

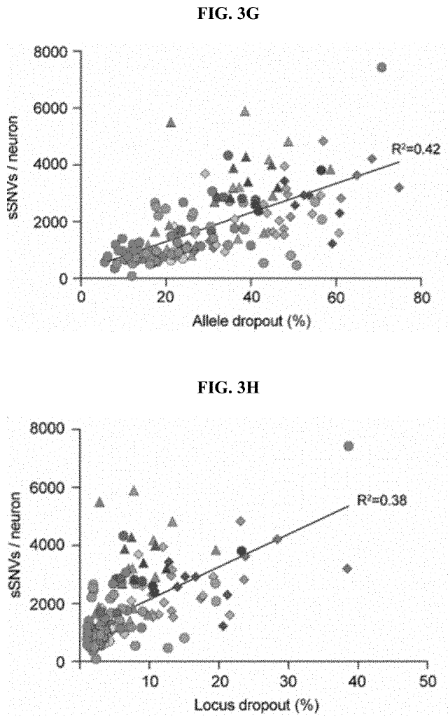

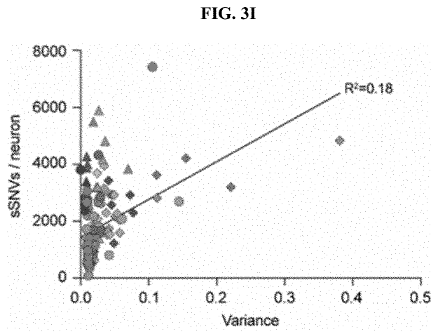

Single neuronal nuclei were isolated using flow cytometry, the nuclei were then lysed on ice in alkaline conditions, as previously performed, to minimize lysis-induced artifacts, and their genomes were then amplified using multiple displacement amplification (MDA). The amplified DNA was then subjected to 45×WGS (FIG. 1A) (G. D. Evrony et al., Cell 151, 483-496 (2012); M. A. Lodato et al., Science 350, 94-98 (2015)). To identify somatic single nucleotide variants (sSNVs) with high confidence, a bioinformatic pipeline was developed, called Linked-Read Analysis (LiRA), to delineate true double-stranded sSNVs from single-stranded variants and artifacts (C. L. Bohrson et al., bioRxiv, (2017)). This method employs read-based linkage of candidate sSNVs with nearby germline SNPs and performs a model-based extrapolation of the genome-wide mutational frequency based on the ˜20% of sSNVs that are sufficiently close to germline SNPs (FIG. 1B). Importantly, sSNVs determined by this algorithm showed alternate allele frequency distribution strikingly matching that of the germline SNVs (FIG. 2A, FIG. 2B). sSNV counts were not systematically influenced by technical metrics, such as post-mortem interval, time in storage, and coverage uniformity (FIG. 3A, FIG. 3B, FIG. 3C, FIG. 3D, FIG. 3E, FIG. 3F, FIG. 3G, FIG. 3H, and FIG. 3I).

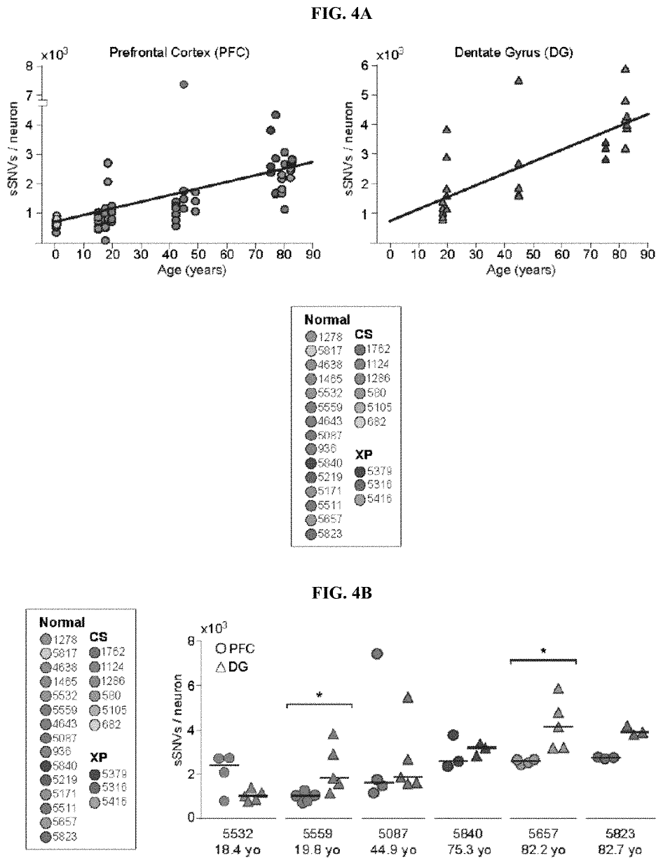

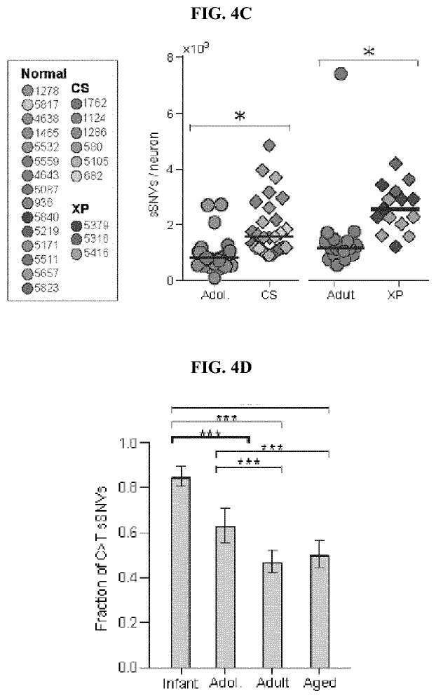

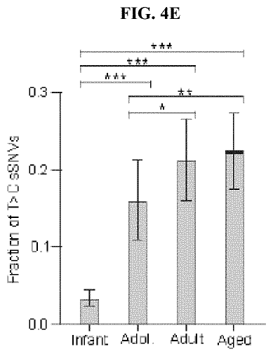

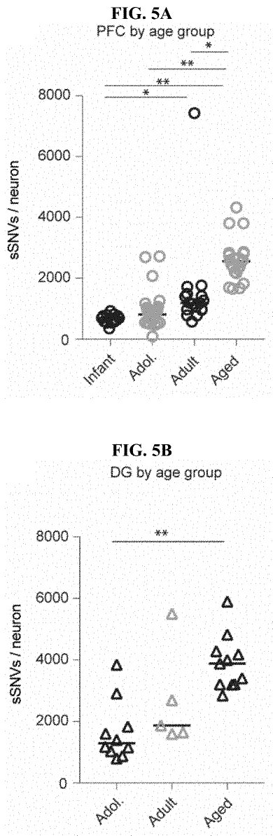

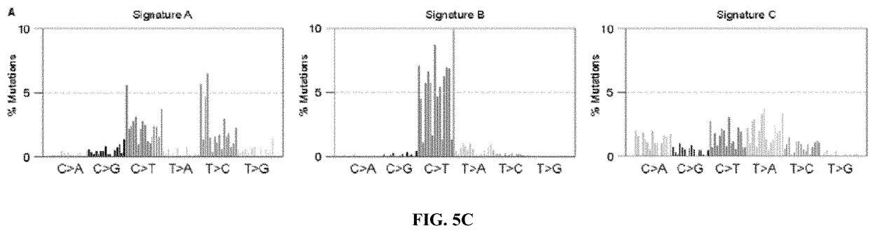

Across all normal neurons, genome-wide sSNV counts correlated with age (FIG. 4A, FIG. 5A, FIG. 5B, and FIG. 5C) (p=9×10⁻¹², mixed effects model, see Materials and Methods, below), despite some within-individual and within-age group heterogeneity. To explore potential variation in different brain regions, matched DG and PFC neurons were sequenced in six cases (FIG. 4A, FIG. 5A, FIG. 5B, and FIG. 5C). The experiments of this example uncovered region-specific sSNV accumulation with age in both PFC (p=4×10⁻⁵) and DG single neurons (p=2×10⁻⁷), suggesting an almost two-fold increase in the rate of accumulation in DG (˜40 sSNVs/year) relative to PFC (˜23 sSNVs/year) (p=8×10⁻⁴). Among the six cases, two had significant increases in DG, three had nominally increased counts in DG compared to PFC, and one had a nominally higher count in PFC (FIG. 4B).

Neurons from postmortem brains of Cockayne syndrome (CS) individuals showed a ˜2.3-fold excess of sSNVs relative to the expected age-adjusted normal PFC rate, and Xeroderma pigmentosum (XP) neurons showed a ˜2.5-fold increase (p=0.006 for both) (FIG. 4C). Progeroid neurons showed a similar number of sSNVs as neurons from aged normal PFC, suggesting that defective nucleotide excision repair accelerated aging via sSNV accumulation.

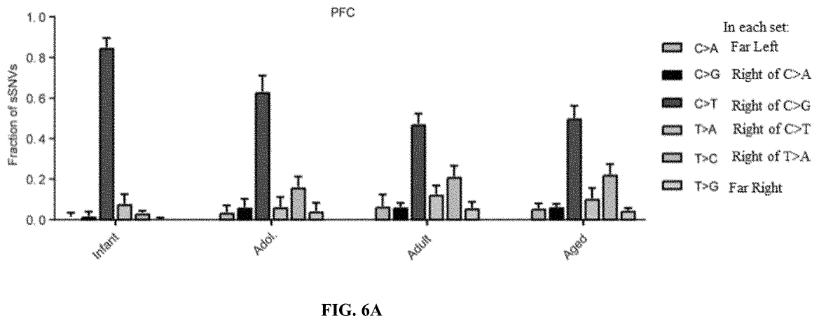

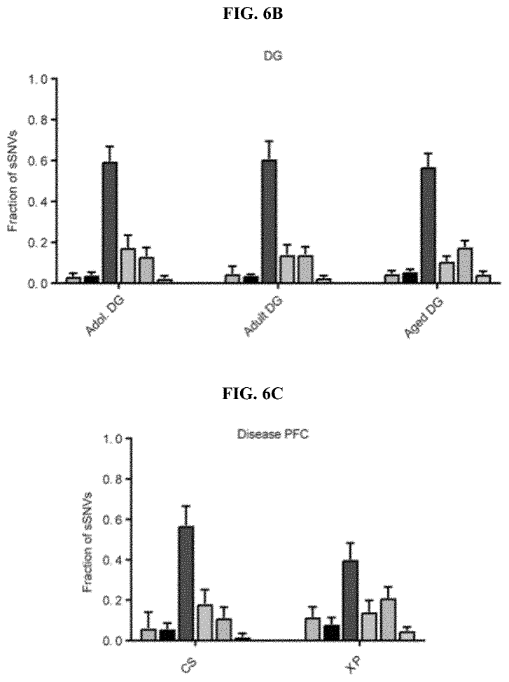

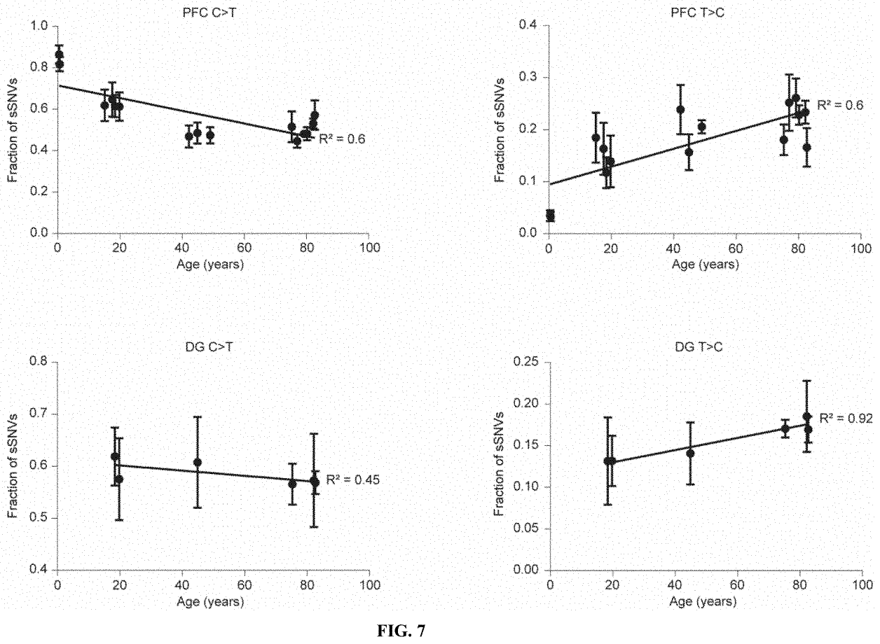

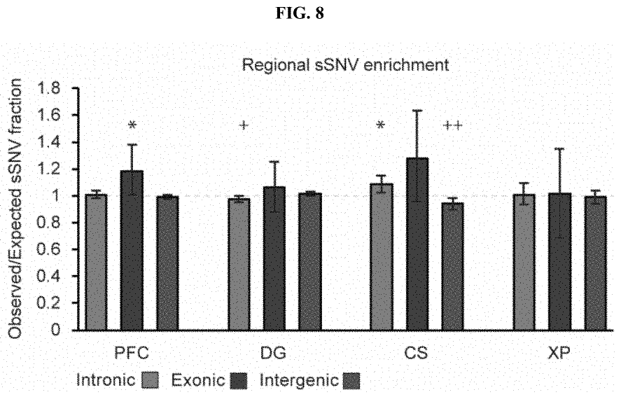

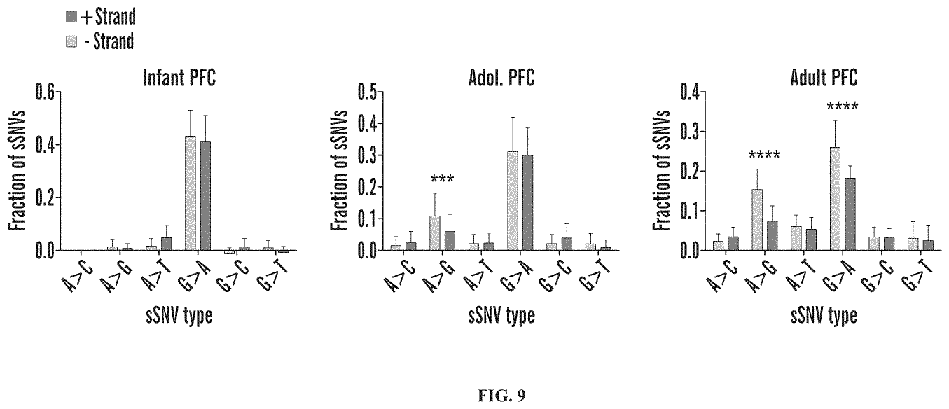

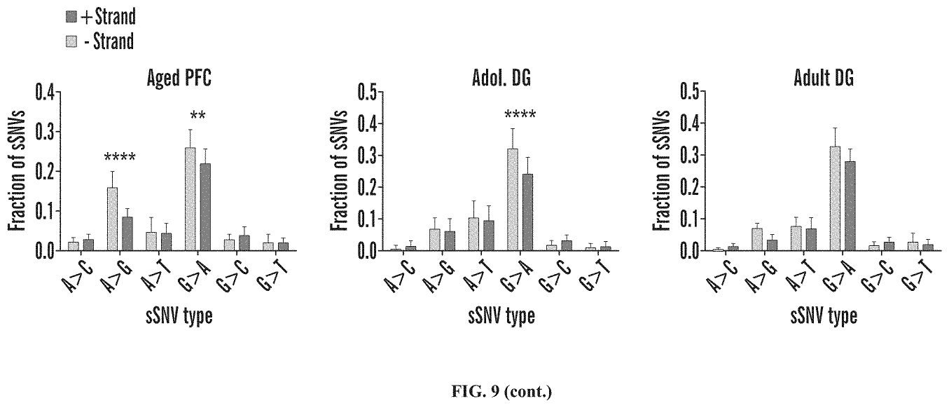

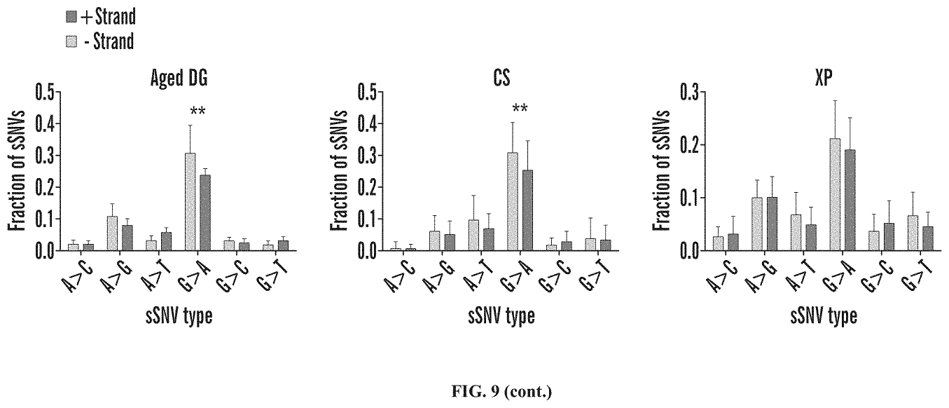

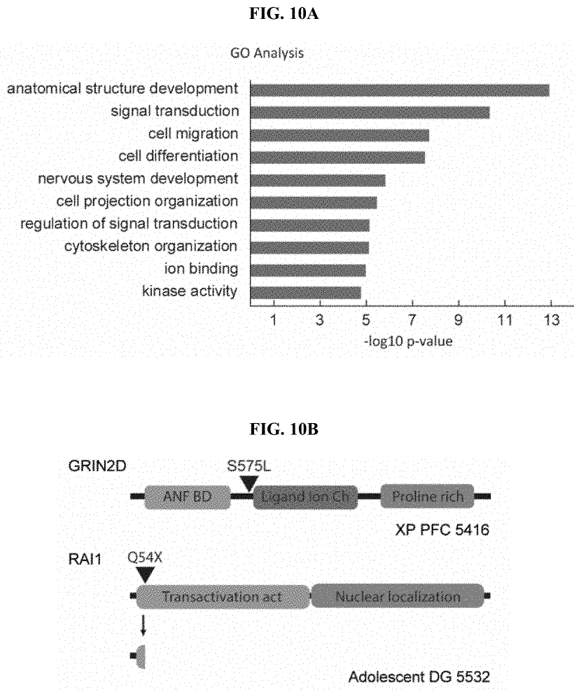

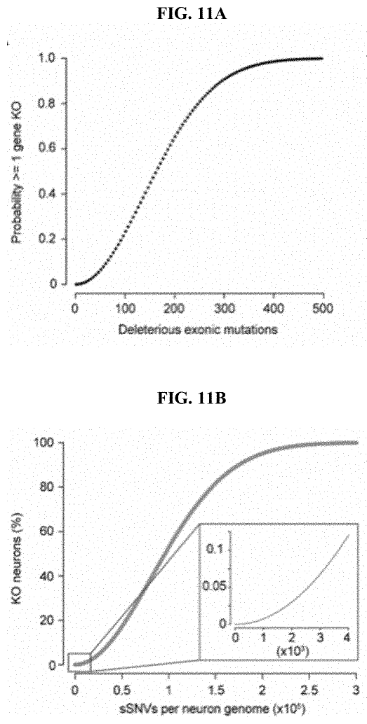

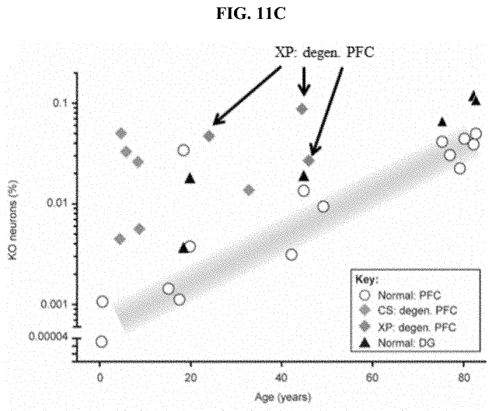

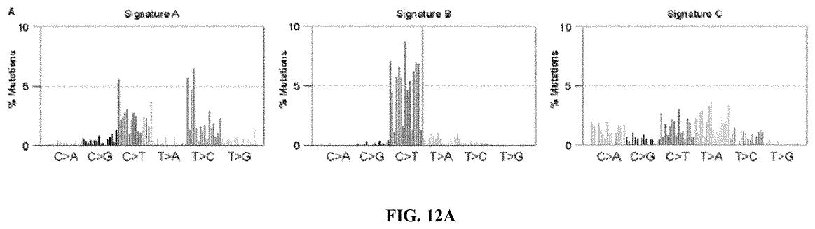

Molecular patterns of sSNVs also evolved with age. Cytosine deamination was previously reported to influence patterns of human neuron sSNVs, resulting in abundant C>T mutations (M. A. Lodato et al., Science 350, 94-98 (2015)). In the experiments of this example, C>T sSNVs accounted for most variants in the youngest PFC samples, but this fraction decreased with age (FIG. 4D, FIG. 6A, FIG. 6B, FIG. 6C, FIG. 7). C>T mutations, while common in many biological contexts, are also a known artifact of MDA (E. T. Lim et al., Nat Neurosci 20, 1217-1224 (2017); Y. Hou et al., Cell 148, 873-885 (2012)). Systematic differences in C>T burden during aging suggest that C>T variants are largely biological and not technical in nature. T>C variants increased in the PFC with age (FIG. 4E, FIG. 6A, FIG. 6B, FIG. 6C, FIG. 7), possibly representing DNA damage linked to fatty-acid oxidation (R. De Bont et al., Mutagenesis 19, 169-185 (2004)). As demonstrated previously (M. A. Lodato et al., Science 350, 94-98 (2015)), neuronal sSNVs in normal PFC were enriched in coding exons (FIG. 8), displayed a transcriptional strand bias (FIG. 9), and genes involved in neural function were enriched for neuronal sSNVs (FIG. 10A, FIG. 10B). Without being bound by theory, coincident probability modeling indicated the linear accumulation of sSNVs in the dataset of this example would result in an exponential accumulation of biallelic deleterious coding mutations, in agreement with classical hypotheses regarding the relationship between mutation and aging (FIG. 11A, FIG. 11B, and FIG. 11C), exacerbating differences in sSNV load in aging, across brain regions, and in disease (L. Szilard, Proc Natl Acad Sci USA 45, 30-45 (1959)).

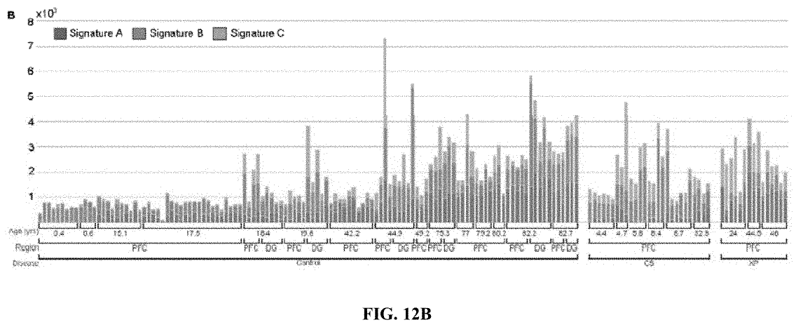

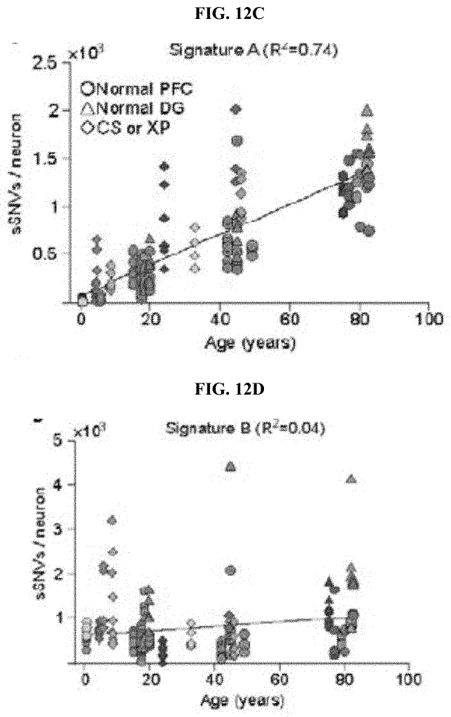

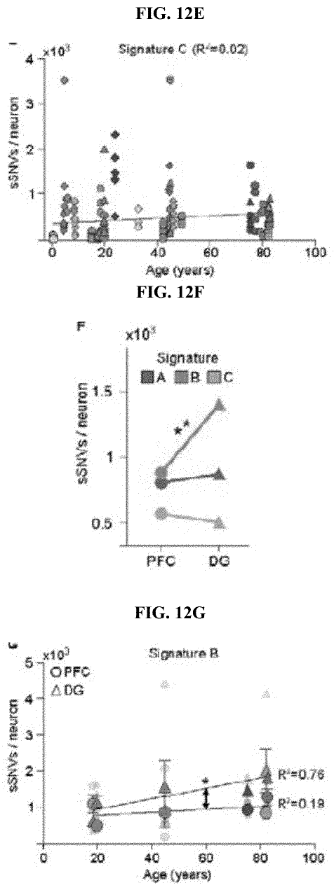

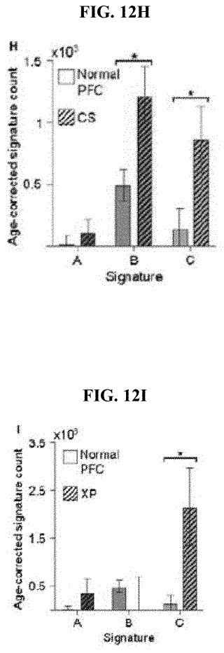

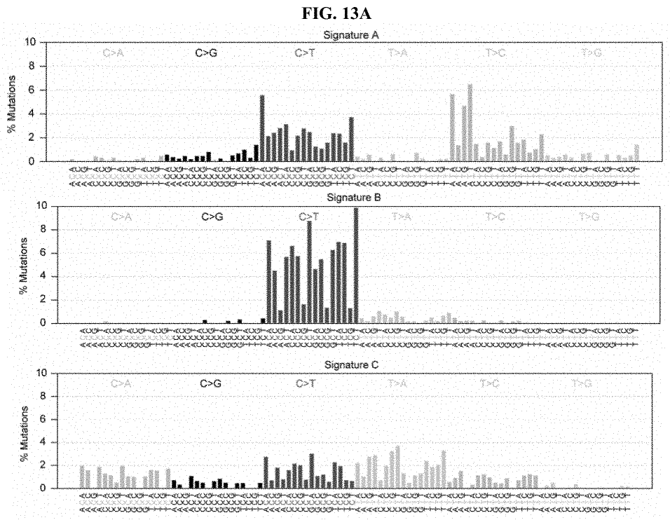

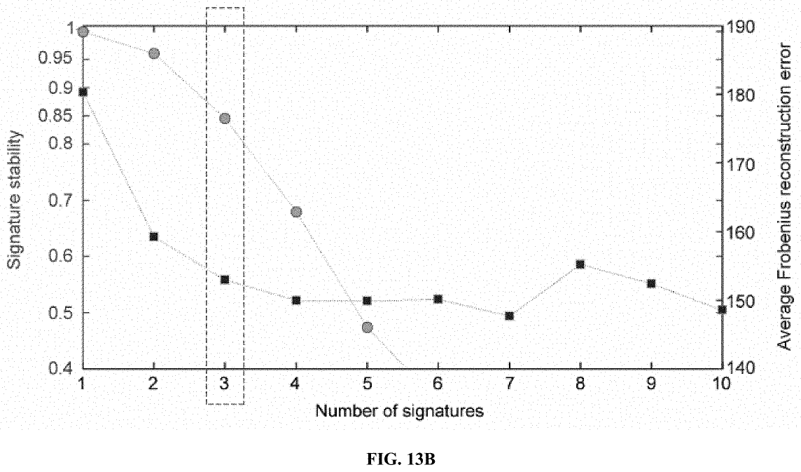

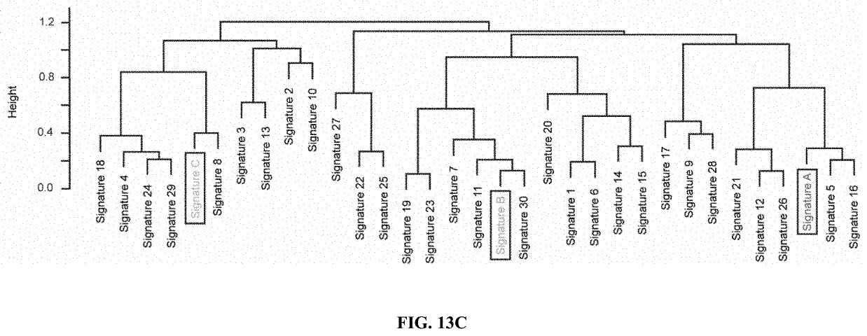

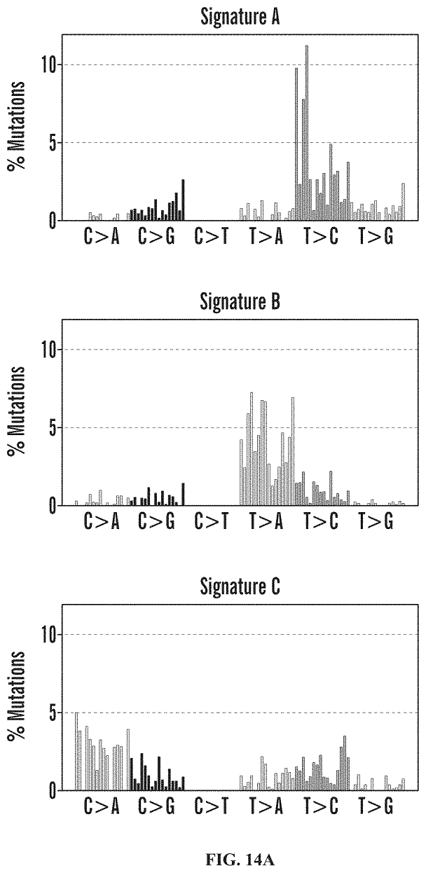

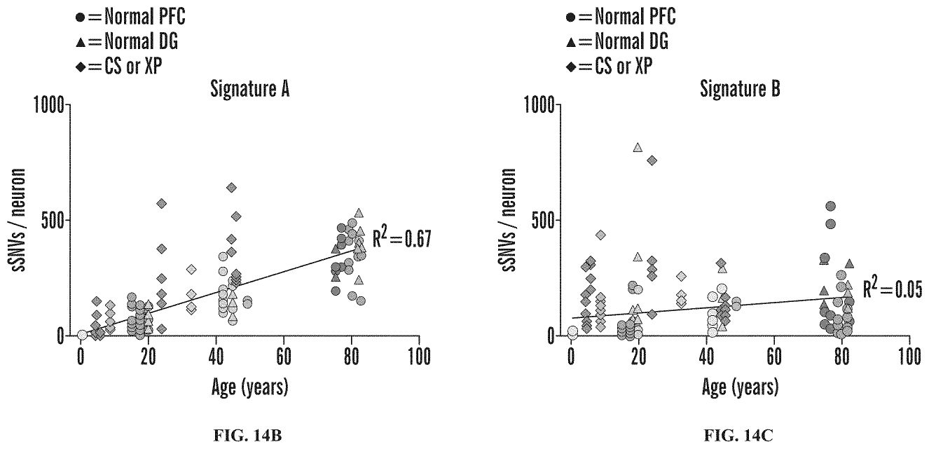

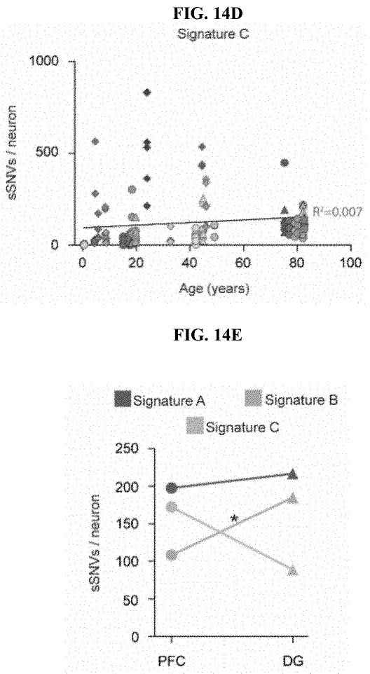

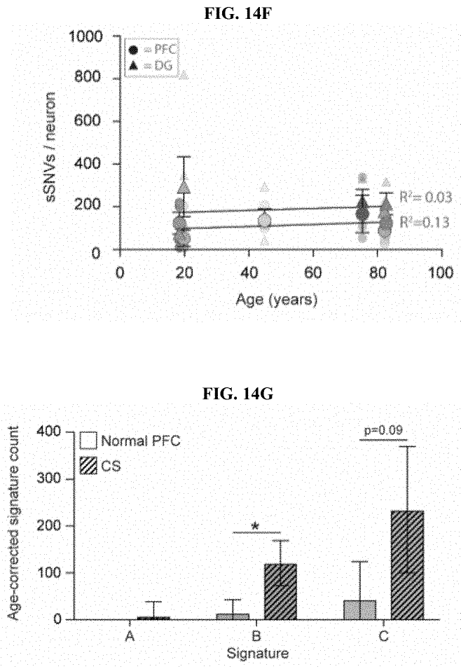

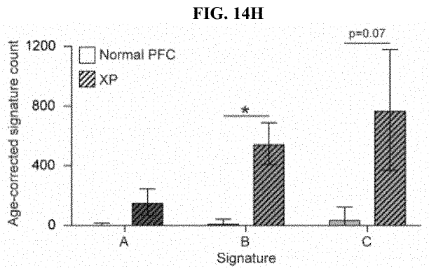

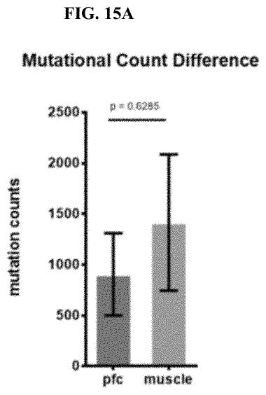

Mutational signature analysis (L. B. Alexandrov et al., Nat Genet 47, 1402-1407 (2015)) revealed three signatures driving single neuron mutational spectra (FIG. 12A, FIG. 12B, FIG. 13A, FIG. 13B, FIG. 13C, FIG. 14A, FIG. 14B, FIG. 14C, FIG. 14D, FIG. 14E, FIG. 14G, FIG. 14H). Signature A was comprised mainly of C>T and T>C mutations and was the only signature to increase with age (p=9×10¹²) independent of brain region or disease status (FIG. 12A, FIG. 12B, FIG. 12C). Signature A resembled a “clock-like” signature found in nearly all samples in a large-scale cancer genome analysis (Signature 5′) (L. B. Alexandrov et al., Nat Genet 47, 1402-1407 (2015)) (FIG. 13A). Data from the experiments of this example show for the first time that a similar clock-like signature was also active in postmitotic cells and hence independent of DNA replication.

Signature B consisted primarily of C>T mutations and did not correlate with age (FIG. 12D), which suggested a mutational mechanism active at very young ages, perhaps prenatally. Signature B may include technical artifacts, which are primarily C>T, but bona fide clonal sSNVs are also predominantly C>T (M. L. Hoang et al., Proc Natl Acad Sci USA 113, 9846-9851 (2016); G. D. Evrony et al., Cell 151, 483-496 (2012)). This signature was enriched in DG compared to PFC (p=2×10⁻⁴) (FIG. 12E, FIG. 12F), and increased with age in DG, but not in PFC (p=0.04, difference in slopes) (FIG. 12G). The observable difference in Signature B between these brain regions, and its correlation with age in DG alone, suggested that it was dominated by a biological mechanism, and these PFC-DG differences strikingly mirror differences in neurogenesis.

A third signature, Signature C (FIG. 13A), was distinct from Signatures A and B by the presence of C>A variants, the mutation class most closely associated with oxidative DNA damage (R. De Bont et al., Mutagenesis 19, 169-185 (2004)). Indeed, CS and XP neurons, defective in DDR, were enriched for Signature C (p=0.016 and 0.023, respectively) (FIG. 12H, FIG. 12I), while Signature C also increased modestly with age in normal neurons (p=0.03). An outlier 5087 PFC neuron with the highest sSNV rate in the data set had a high proportion of Signature C mutations relative to other normal neurons, highlighting that even within a normal brain, some neurons may be subject to catastrophic oxidative damage.

In summary, sSNVs accumulated slowly but inexorably with age in the normal human brain, a phenomenon termed in this disclosure as “genosenium,” and more rapidly still in progeroid neurodegeneration. Within one year of birth, postmitotic neurons already have ˜300-900 sSNVs. Three signatures were associated with mutational processes in human neurons: (1) a postmitotic, clock-like signature of aging; (2) a possibly developmental signature that varied across brain regions; and (3) a disease- and age-specific signature of oxidation and defective DNA damage repair. The increase of oxidative mutations in aging and in disease presents a potential target for therapeutic intervention. Further, elucidating the mechanistic basis of the clock-like accumulation of mutations across brain regions and other tissues has the potential to increase knowledge of age-related disease and cognitive decline. CS and XP cause neurodegeneration associated with higher rates of sSNVs, and it will be important to define how other, more common causes of neurodegeneration may influence genosenium as well.

### Example 2: Patterns of Somatic Mutation Differ Across Tissues

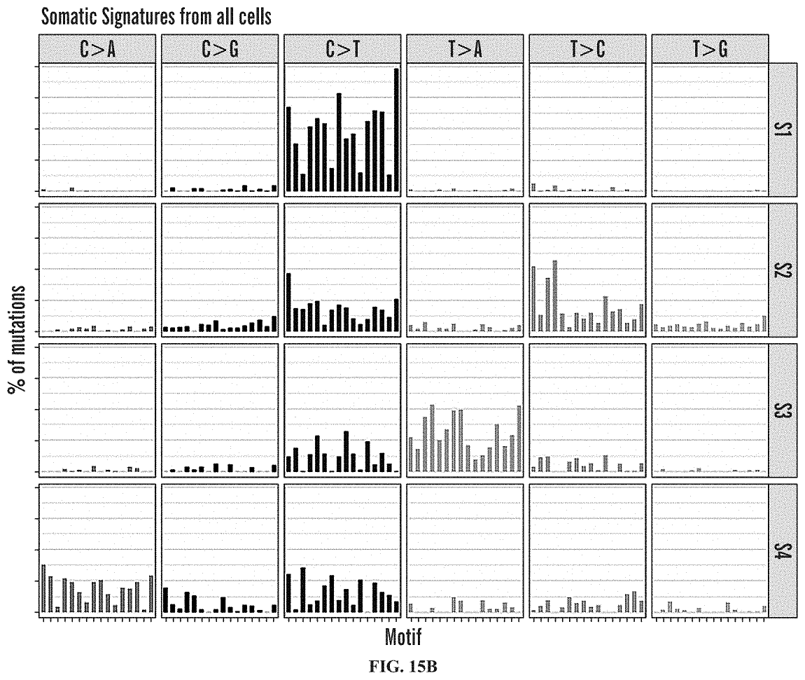

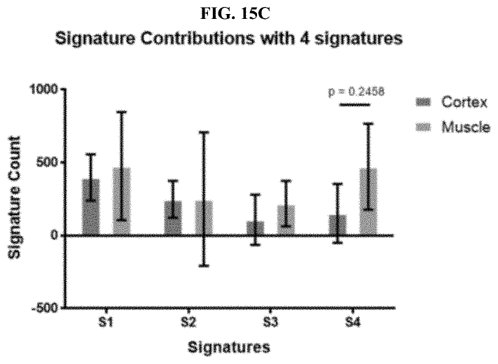

The experiments of this example demonstrated that patterns of somatic mutation were different across tissues. In FIG. 15A, a graph is shown comparing the somatic SNV counts per cell in adolescent cortical neurons (ages 15-19) and muscle cells from two individuals (ages 17 and 20). The results of FIG. 15A show that muscle nuclei have more mutations than PFC nuclei. A molecular signature analysis of mutations discovered across a large dataset of neurons and muscle cells identified four signatures, designated as 51, S2, S3, and S4 in FIG. 15B. Signature 4 was significantly different between muscle and brain, with nuclei from muscle cells having more Signature 4 mutations than brain cell nuclei. (See FIG. 15C). Taken together, the experiments of this example suggest that somatic mutations provide a record of tissue-relevant cellular damage that can distinguish different cell types.

The results described herein above, were obtained using the following methods and materials.

### Human Tissues and DNA Samples

All human tissues were obtained from the NIH NeuroBioBank at the University of Maryland. Frozen post-mortem tissues from three neurologically normal individuals, UMB4638, UMB1465, and UMB4643, were obtained as part of a previous study (G. D. Evrony et al., Cell 151, 483-496 (2012)). Samples were processed according to a standardized protocol (http://medschool.umaryland.edu/btbank/method2.asp) under the supervision of the NIH NeuroBioBank ethics guidelines. Bulk DNA was extracted using the QIAamp DNA Mini kit with RNase A treatment. The isolation of single nuclei using fluorescence-activated nuclear sorting (FANS) for NeuN and their whole-genome amplification using multiple displacement amplification (MDA) were described previously (G. D. Evrony et al., Cell 151, 483-496 (2012); F. B. Dean et al., Genome Res 11, 1095-1099 (2001); G. D. Evrony et al., Neuron 85, 49-59 (2015)). Briefly, nuclei were prepared from fresh frozen human brain tissue samples, previously stored at −80° C., in a dounce homogenizer using a chilled nuclear lysis buffer (10 mM Tris-HCl, 0.32M Sucrose, 3 mM Mg (Acetate)₂, 5 mM CaCl₂, 0.1 mM EDTA, pH 8, 1 mM DTT, 0.1% Triton X-100) on ice. Tissue lysates were carefully layered on top of a sucrose cushion buffer (1.8M Sucrose, 3 mM Mg (Acetate)₂, 10 mM Tris-HCl, pH 8, 1 mM DTT) and ultra-centrifuged for 1-2 hours at 30,000 G. Nuclear pellets were resuspended in ice-cold PBS supplemented with 3 mM MgCl₂, filtered, then stained with an anti-NeuN antibody (Millipore MAB377). Nuclei were then sorted by flow cytometry using a custom sheath fluid (1×PBS with 3 mM MgCl₂), one nucleus per well into 384- or 96-well plates with each well containing 2.8 μl alkaline nuclear lysis buffer (200 mM KOH, 5 mM EDTA, 40 mM DTT) prechilled on ice. Nuclei were lysed on ice for 15-30 minutes, and then neutralized on ice in 1.4 μl of neutralization buffer (400 mM HCl, 600 mM Tris-HCl, pH 7.5). Then, multiple displacement amplification (MDA) was performed in a 20 μl total reaction volume by addition of an MDA master mix (2 μl 10×Phi29 reaction buffer (Epicentre), 8.4 μl H₂O, 4 μl 10 mM dNTP, 1 μl 1 mM random hexamer (5′ dNdNdNdN*dN*dN-3′ [where*=thiophosophate linkage])(IDT or Thermo-Fisher), 0.4 μl repliPHI polymerase (40 U) (Epicentre)). MDA was performed at 30° C. for 16 hours. This protocol was used for all samples in Example 1, including previously published and new samples.

For the experiments of Example 2, the same protocol as disclosed above was used, except that for the fluorescence-activated nuclear sorting (FANS) in muscle, the nuclei were stained with an anti-Desmin antibody (Abcam 243M (Clone D33)), instead of the anti-NeuN antibody.

### Library Preparation and Whole Genome Sequencing

As described (G. D. Evrony et al., Cell 151, 483-496 (2012); G. D. Evrony et al., Neuron 85, 49-59 (2015)), for previously published 1465 single neurons, 500 ng of amplified DNA from each neuron was sheared on a Covaris E210 focused ultra-sonicator. Paired-end barcoded whole genome sequencing (WGS) libraries were prepared with the NEXTflex DNA sequencing kit (Bioo Scientific) with 8 cycles of PCR amplification. Paired-end sequencing (100 bp×2 or 101 bp×2) was performed on Illumina HiSeq 2000 sequencers at the Harvard Biopolymers Facility (Harvard Medical School, Boston Mass.) and Axeq (Seoul, South Korea). For 4638 and 4643 single neurons, also previously published (M. A. Lodato et al., Science 350, 94-98 (2015)), 100 ng of DNA from each neuron was sheared on a Covaris Ultra-Sonicator into ˜350 bp fragments. Paired-end barcoded WGS libraries were then prepared with the Illumina TruSeq Nano LT sample preparation kit. Paired end sequencing (150 bp×2) was performed on a HiSeq X10 instrument. Library preparation and sequencing were performed at the New York Genome Center, New York, N.Y. Sequencing reads are deposited in the NCBI SRA with accession numbers SRP041470 (UMB1465) and SRP061939 (UMB4638 and UMB4643). For novel datasets generated for this study, MDA-amplified DNA was sheared and libraries were generated by Macrogen Genomics using Illumina Tru-Seq Kits and using an Illumina HiSeq X10 instrument (FIG. 17).

### Read Mapping and BAM File Generation

Reads generated from WGS were mapped onto the human reference genome (GRCh37 with decoy) by BWA (ver. 0.7.8) (H. Li, R. Durbin Bioinformatics 25, 1754-1760 (2009)) with default parameters. Duplicate reads were marked by MarkDuplicates of Picard tools (ver. 1.130) and then post-processed with local realignment around indels and base quality score recalibration using Genome Analysis Toolkit (GATK) (ver. 3.4) (A. McKenna et al., Genome Research 20, 1297-1303 (2010)).

### Somatic Single-Nucleotide Variant Calling

To obtain an initial set of variant calls, GATK's HaplotypeCaller (HC) (ver. 3.4) gVCFs were created for each bulk and single cell sample using ‘emitRefConfidence’ mode. Subsequently, the HC's GenotypeGVCFs tool was used to jointly call variants across all of the samples. The resulting call set provided the first putative set of germline and somatic mutations in the samples for the downstream linkage analysis.

A custom tool was created (C. L. Bohrson et al., bioRxiv, (2017)) for determining paired-end, read-backed linkage between germline sites and candidate somatic mutations detected by GATK. Only sites showing strong evidence for only two haplotypes were considered. Subsequently our power to detect mutations was calculated for each cell to correct for sensitivity and used to extrapolate a genome-wide mutation burden from the number of variants initially detected. Power was calculated by determining the number of sites on either or both alleles that were linkable to any common, heterozygous germline locus based on the reads of that particular cell. This was then adjusted to account for the estimated false-positive rate and confidence values, yielding an adjusted estimated mutation count for each sample.

### Analysis of Dropout and Allelic Imbalance

MDA has been shown to be susceptible to allelic and locus dropout in single cell sequences (Y. Hou et al., Cell 148, 873-885 (2012)). The former refers to the loss of one copy of an allele in the sequencing data, and the latter refers to the complete loss of reads spanning a given locus in the genome. To calculate our allelic dropout rate, a set of germline sites called as heterozygous were first considered in each bulk tissue sample that was also listed as common (minor allele frequency >1%) variants in the dbSNP database (S. T. Sherry et al., Nucleic Acids Res 29, 308-311 (2001)).

After confirming that the variant allele was observed in the bulk BAM, a samtools (ver. 1.3) (http://samtools.sourceforge.net/) pileup was created for each single cell using default settings. Any site where either the reference or variant germline allele seen in the matched bulk sample was not observed was considered an incidence of allelic dropout. The percent of common germline variants missing in each cell was then calculated. To account for locus dropout, every base position was recorded in the genome where there was coverage in the bulk tissue sequence of each individual. The same was done for each single cell and all the genomic positions at which reads were present in the matched bulk were determined, but not the single cell sequence. The percent of positions missing any reads was then calculated as the locus dropout count for each cell.

### Analysis of Relationship Between Quality Control Metrics and Somatic SNV Counts

To gain insight into whether the number of somatic mutations observed were related to the quality of the cells sequenced, a wide range of QC metrics were compared in each cell to that cell's estimated sSNV count. Metrics considered were: post-mortem interval (PMI) before preservation, years in storage, RNA integrity number (RIN), sex, estimated MDA amplicon size, total number of GATK HC variants called, allelic dropout rate, locus dropout rate, and spectral density variance (M. A. Sherman et al., bioRxiv, (2017)). PMI, number of years in storage, and RIN are all uncorrelated with the estimated somatic SNV rate with adjusted R² values of >0.1 (0.14, 0.06, and 0.05 respectively) suggesting the overall quality of the tissues did not affect the estimated sSNV counts. The estimated amplicon size of the MDA fragments was also uncorrelated with the estimated somatic sSNV count (R²=0.08).

Estimates of the error rate of phi29 (M. A. Lodato et al., Science 350, 94-98 (2015); J. Wang et al., Cell 150, 402-412 (2012); C. F. de Bourcy et al., PLoS One 9, e105585 (2014)) compared to previous estimates of the sSNV count (M. A. Lodato et al., Science 350, 94-98 (2015); J. L. Hazen et al., Neuron 89, 1223-1236 (2016); M. Lynch, Proc Natl Acad Sci USA 107, 961-968 (2010)) suggest, that in a given unfiltered single-cell WGS dataset, artifacts should vastly outnumber true mutations. Unfiltered calls from GATK were used as the input for the linkage analysis. Thus, the relationship between linkage-based sSNV rate estimates and the total number of GATK HC variants called is likely a highly relevant quality control metric to consider. If the number of GATK HC calls correlated positively with linkage-based somatic sSNV estimates, it might suggest that a large fraction of the signal might derive from artifacts. However, the correlation between the total GATK calls and its estimated SNV count is actually negative with an R² of 0.14 (FIG. 3A, FIG. 3B, FIG. 3C, FIG. 3D, FIG. 3E, FIG. 3F, FIG. 3G, FIG. 3H, and FIG. 3I) implying the linkage-based pipeline was identifying true somatic SNVs from the set and not simply reducing the set of GATK variants passed into it.

The allelic dropout rate, locus dropout rate, and spectral density variance were all weakly correlated with estimated somatic SNV counts in the cells (R² of 0.42, 0.38, and 0.18, respectively) (FIG. 3A, FIG. 3B, FIG. 3C, FIG. 3D, FIG. 3E, FIG. 3F, FIG. 3G, FIG. 3H, and FIG. 3I). Importantly, these correlations were weaker than the correlation between estimated somatic sSNV count and age. Moreover, DNA lesions can inhibit various nucleic acid polymerases that rely on intact DNA templates, including endogenous DNA polymerases during DNA replication (K. Baynton, R. P. Fuchs, Trends Biochem Sci 25, 74-79 (2000)), endogenous RNA polymerase II (Y. Morita, S. Iwai, I. Kuraoka, J Toxicol Sci 36, 515-521 (2011)), and enzymes used in in vitro settings such as T7 RNA polymerase (Y. Sonohara, et al., Genes Environ 37, 8 (2015)), Taq polymerase (M. M. Jennerwein, A. Eastman, Nucleic Acids Res 19, 6209-6214 (1991)), and, most relevantly the phi29 polymerase used in MDA (T. Woyke et al., PLoS One 6, e26161 (2011)). This inhibition results in polymerase stalling, which, if left unresolved, results in a blockage of amplification of the damaged template. The result of such blocked amplification in the context of a WGS experiment would likely be dropout of the damaged DNA fragment, and a lower measured copy number of this locus in the final dataset. Thus, samples with increased levels of endogenous DNA lesions might have increased levels of allelic dropout, locus dropout, and copy number variance. Elevated levels of lesions are considered to be expected in the aging human brain (T. Lu et al., Nature 429, 883-891 (2004)) and both CS and XP patients (J. A. Marteijn, et al., Nat Rev Mol Cell Biol 15, 465-481 (2014); K. Diderich, et al., DNA Repair (Amst) 10, 772-780 (2011)), samples were also estimated to have more bona fide, double-stranded mutations likely due to this damage. It is perhaps unsurprising, then, that increased levels of allelic dropout were observed, locus dropout, and copy number variance increasing alongside the somatic mutation count.

### Mixed-Effect Modeling to Compare Mutational and Signature Burdens

Since mutational burden in neurons from the same donor and tissue may be correlated due to shared biological environment, all tests for the effects of age and differences between tissues and healthy and disease states were carried out in a linear mixed-effects regression framework. Covariates of interest (e.g., age, tissue, disease status) were modeled as fixed effects as in standard ordinary least squares regression while the clustering of neurons from the same donor-tissue pair were modeled as random effects. This framework allows for accurate estimates of the means, variances, and significances of each covariate while accounting for the increased uncertainty due to clustering of samples from the same biological origin. A t-test using the Satterthwaite approximation on the degrees of freedom was used to test each covariate for difference from zero, with a p-value<0.05 considered significant. The model for each comparison presented in the text is described below. All p-values presented in this disclosure derive from this modeling, unless otherwise indicated.

To test the differential effect of age in PFC and DG, a model was fitted of the form yᵢⱼₖ=(β+γᵢ)*age+μ+Uᵢⱼ+ϵᵢⱼₖ where yᵢⱼₖ is sample k from donor and tissue i, β is the fixed-effect of age, γᵢ is the fixed-effect of each tissue on age, μ is the number of sSNVs at birth, and Uᵢⱼ˜N(0,σᵣ²) is the random effect of the donor-tissue pair, and ϵᵢⱼₖ˜N(σ²) is the measurement error of each sample. Additionally it was confirmed that there was no significant difference between the number of sSNVs at birth in DG and PFC neurons by fitting the model yᵢⱼₖ=(β+γᵢ)*age+αᵢ+μ+Uᵢⱼ+ϵᵢⱼₖ where αₗ is the tissue-specific contribution of sSNVs at birth. Note, that both the estimate of number of sSNVs at birth (718; CI: 176-1268) and the conclusion that there was no difference in this number between brain regions is highly consistent with the conclusions presented in Bae et al., Science 7 Dec. 2017 (available online at http://science.sciencemag.org/content/early/2017/12/06/science.aan8690.long).

To test the difference in means between age groups within the same tissue, the model yᵢⱼₖ=αᵢ+μ+Uⱼ+ϵᵢⱼₖ was fitted to all age groups simultaneously where αᵢ is the mean difference between age groups relative to the global mean μ and Uⱼ is the random-effect of donor. To test the difference in means within an age group across tissues the model yᵢⱼₖ+αᵢ+μ+Uᵢⱼ+ϵᵢⱼₖ was fitted to each age-tissue combination where a is the mean difference between tissues and Uᵢⱼ is the random-effect of each donor-tissue pair.

To test the difference between healthy and disease (CS or XP) PFC samples in an age-controlled manner, the model yᵢⱼₖ=β*age+αᵢ+μ+Uⱼ+ϵᵢⱼₖ was fitted, where β is the effect of age, is the effect of disease state (none, CS, or XP), and Uⱼ and ϵᵢⱼₖ are as previously. Testing the age-controlled mean difference between disease states is equivalent to testing for a significant difference between the αᵢ.

To test the effect of signatures both across tissues and across disease states, a hierarchical model of the form yᵢⱼₖ=(β+γᵢ+δⱼ)*age+αᵢ+ηⱼ+μ+Uᵢⱼ+ϵᵢⱼₖ where yᵢⱼₖ was fitted, where yᵢⱼₖₗ is the estimated mutational burden in sample l of donor k with disease status j from tissue i, β is the global effect of age, γᵢ is the additional age-related effect of tissue, δⱼ is the additional age-related effect of disease, αᵢ is the age-corrected mean effect of tissue, ηⱼ is the age-corrected mean effect of tissue, μ is as previously, Uᵢₖ is the random-effect of each donor-tissue pair (note, since no donor belongs to both a disease and non-disease state, we do not have to stratify on disease state), and ϵᵢⱼₖₗ is as previously. Testing for the global effect of age is equivalent to testing if β is different than zero while testing for tissue and disease-specific age-related effects amounts to testing for γᵢ and δⱼ different than zero, respectively. Testing for age-controlled differences between tissues is equivalent to testing for differences between the αᵢ and testing for differences between disease states is equivalent to testing for differences between the ηⱼ. The effect of age was also tested across all samples with the model yᵢⱼₖₗ=β·age+ϵᵢⱼₖₗ, and an age-independent mean difference was also tested for between tissues by removing γᵢ term from the hierarchical model and retesting for a significant difference between the αᵢ.

All models were fit using the function lmer from the lme4 R package (v 1.1-14) and the lmerTest package (v 2.0-33) to perform the Satterthwaite corrected t-tests.

### Strand Bias Analysis

True mutations in transcribed regions of the genome display a signature of transcriptional strand bias (P. Green et al., Nat Genet 33, 514-517 (2003); P. Polak et al., Genome Res 18, 1216-1223 (2008)), resulting from asymmetric repair of coding and non-coding strands. To test for strand bias in sSNVs, the UCSC table browser was used to find all RefGene transcripts associated with single-neuron sSNVs. Only sSNVs that had known transcripts all going in the same direction were considered. Transcriptional directions to sSNVs that overlapped a transcript were tallied using numbers and the data was collapsed to only report one complement of each base pair.

Enrichment of sSNVs in Genomic Features

Annotations from the Homo. sapiens (Team BC) package version 1.3.1 in R were used to identify somatic SNVs falling within exons, introns, and intergenic regions. For each region of interest, the number of sSNVs were counted in the corresponding group (dentate gyrus, prefrontal cortex, cockayne syndrome, and xeroderma pigmentosum) appearing in the region across cells and computed the expected number (assuming no enrichment) using the proportion of callable sites available in each region and the total number of sSNVs across regions. To derive confidence intervals for the value of observed/expected for each feature, bootstrapping was performed with 10 million iterations on data from individual cells in each group using the boot (version 1.3-20) (A. C. Davison et al. (Cambridge University Press, Cambridge; New York, N.Y., USA, 1997), pp. x, 582) package in R (‘boot’ function). To account for testing 3 regions simultaneously, confidence intervals were derived (boot.ci′ function with type=“bca”) at the 0.05/3≈0.167 and 0.001/3≈0.003 significance levels. If the confidence interval for a feature did not include 1, the enrichment or depletion of the feature was reported as significant (p<0.05 or p<0.001).

### Signature Analysis

To discover signatures of mutational processes, the frequency of mutations were calculated in their context for a trinucleotide substitution matrix for 161 neurons from the identified single-neuron sSNVs. Mutation signatures were detected by fitting nonnegative matrix factorization-based mutational signature framework (L. B. Alexandrov et al., Cell Rep 3, 246-259 (2013)). As the number of signatures were increased from one to ten, the signature stability and reconstruction error was estimated of each signature and three signatures were chosen (Signature A, B, and C) that maximize the number of identified signatures while minimizing error (FIG. 13A, FIG. 13B, and FIG. 13C). The identified three signatures were clustered with the reported 30 signatures that can be found at COSMIC website (http://cancer.sanger.ac.uk/cosmic/signatures), using unsupervised hierarchical clustering using correlation as the distance metric (FIG. 13A, FIG. 13B, and FIG. 13C). Using the contribution of each signature to each neuron extracted by the framework, the correlation of signatures with age was estimated.

### Functional Enrichment Analysis

To define pathways enriched for sSNVs in our dataset, a modified version of the GOseq algorithm (M. D. Young et al., Genome Biology 11, R14 (2010)) was used to control for both gene length and gene-specific averaged statistical power biases in the assessment of functional enrichment of Gene Ontology (GO) terms (M. Ashburner et al., Nature Genetics 25, 25-29 (2000)). Standard functional enrichment analyses based on the hypergeometric distribution assume that genes have an equal probability of being identified as significant. However, this assumption is violated in the assessment of sequencing data due to length dependencies and the ability to statistically detect somatic variants in different regions of the genome, thus rendering the hypergeometric distribution unsuitable for the Fisher's Exact Test. As previously described, the Wallenius' non-central hypergeometric distribution (A. Fog, Communications in Statistics—Simulation Computation 37, 2410257 (2008)), as implemented in the GOseq algorithm, was therefore used to determine the probability of GO term enrichments for somatic mutation data sets (M. A. Lodato et al., Science 350, 94-98 (2015)). Briefly, annotated gene length information for all annotated genes on Chromosomes 1 to 22 was obtained from BioMart (S. Durinck et al., Nat Protoc 4, 1184-1191 (2009)). Genomic regions over which there was power (please see above) were concatenated and mapped to these same genes. Gene-specific averaged statistical power was then determined. Genes were assigned a binary value (0 or 1) based on mutation status. To control for both gene length and statistical power biases, a probability weighting function was estimated from the data as previously described (M. D. Young et al., Genome Biology 11, R14 (2010)). Functional enrichment was assessed with the Wallenius approximation method as implemented in the GOseq algorithm, and the Benjamini and Hochberg method was used to correct for multiple hypothesis testing (Y. Benjamini et al., Journal of the Royal Statistical Society. Series B (Methodological) 57, 289-300 (1995)). As previously reported (M. A. Lodato et al., Science 350, 94-98 (2015)), nested gene ontology analysis across the dataset revealed that neuronal pathway genes were enriched for somatic mutations, suggesting transcription itself contributes to the neuronal somatic mutation rate (FIG. 10).

### Modeling Accumulation of Gene KOs in PFC Neurons

Although many heterozygous mutations would likely compromise neuronal function (G. R. Crabtree, Trends in Genetics: TIG 29, 1-3 (2013); G. R. Crabtree, Trends Genet 29, 3-5 (2013)), it is expected that biallelic, exonic, deleterious “gene knockout” (KO) mutations in essential genes would be especially damaging, and that there may be a threshold for the accumulation of such KOs above which neuronal function would deteriorate. The accumulation of gene KOs in cortical neurons was estimated using the probability of two or more coincident events, in this case, two deleterious sSNVs within one gene, causing LOF (FIG. 11A, FIG. 11B, and FIG. 11C). Using this function, the probability of observing two mutations in one or more coding genes (where n=number of deleterious mutations and where the number of coding genes=19,000 (I. Ezkurdia et al., Hum Mol Genet 23, 5866-5878 (2014)) is:

$$p ( n ) = 1 - ( 1 \times ( 1 - \frac{1}{19000} ) \times ( 1 - \frac{2}{19000} ) \times … \times ( 1 - \frac{n - 1}{19000} ) ) = 1 - ( \frac{n ! \cdot \frac{19000}{n}}{{19000}^{n}} )$$

This equation can be closely approximated with the exponential function: y==1−e^{-n(n-1)/(2×19000)}. This exponential function can be scaled from deleterious coding mutations to whole genome sSNVs based on the sSNVs identified in this invention. Specifically, 0.76% of somatic sSNVs were found in coding genes, 44% of those were nonsense or predicted deleterious missense variants, and 50% of the time, two mutations in a gene will be on opposite alleles, creating a compound heterozygous LOF. This exponential function can also be scaled to estimate the number of neurons that contain 1 or more LOF genes, “KO neurons,” using the estimate that the human cerebral cortex contains 80,000,000,000 neurons (S. Herculano-Houzel, Front Hum Neurosci 3, 31 (2009)).

While somatic mutation rates accumulated linearly, the probability of coincident events predicts that these KO neurons would in fact increase exponentially, in concordance with a longstanding hypothesis of aging that linear accumulation of somatic mutations causes exponentially increasing tissue dysfunction and mortality (L. Szilard, Proc Natl Acad Sci USA 45, 30-45 (1959); G. Failla, Ann N Y Acad Sci 71, 1124-1140 (1958)).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of identifying a single-nucleotide variant (SNV) in a single cell, the method comprising:
(a) purifying genomic DNA from a single cell;
(b) amplifying the genomic DNA;
(c) sequencing the amplified genomic DNA;
(d) detecting a variant nucleotide on forward and reverse strands, wherein the presence of the variant nucleotide on the forward and reverse strands identifies a double-stranded mutation that is a single-nucleotide variant.

2. The method of claim 1, wherein the single-nucleotide variant is a somatic mutation.

3. The method of claim 1, wherein detecting the variant nucleotide is in proximity to a germline variant.

4. The method of claim 1, wherein the cell is a neuron, cardiac cell, muscle cell, or skin cell.

5. The method of claim 1, wherein the purifying step comprises alkaline lysis on ice or comprises isolating the nucleus from the single cell.

6. The method of claim 1, comprising eliminating from a sequence read a variant nucleotide that is not present on forward and reverse strands, wherein the absence of the variant nucleotide on a forward or reverse strand identifies an error in genomic sequencing.

7. The method of claim 6, wherein the error is a DNA lesion that is biologically induced pre-mortem, biologically induced post-mortem, or generated during DNA purification, nuclear isolation, cell lysis, DNA amplification, DNA library preparation, or DNA sequencing.

8. A method of determining the genomic age of a subject, the method comprising:
(a) purifying genomic DNA from a single cell obtained from the subject;
(b) amplifying the genomic DNA;
(c) sequencing the amplified genomic DNA;
(d) measuring the number of somatic variant nucleotides on forward and reverse strands, wherein the presence of the somatic variant nucleotide on the forward and reverse strands identifies a double-stranded mutation that is a somatic single-nucleotide variant; and
(e) determining a genomic age from the number of somatic variant nucleotides relative to a reference, wherein increased somatic variant nucleotides relative to a reference indicates advanced genomic age.

9. The method of claim 8, comprising measuring the number of somatic variant nucleotides in at least 2, 3, 4, 5 or more cells.

10. The method of claim 8, wherein the somatic variant nucleotide is in proximity to a germline variant.

11. The method of claim 8, wherein the subject has or is identified as having a progeroid disease.

12. The method of claim 11, wherein the progeroid disease is Cockayne syndrome (CS) or Xeroderma pigmentosum (XP).

13. The method of claim 8, comprising measuring the rate of accumulation of genome-wide somatic SNVs.

14. A method of measuring the rate of accumulation of genome-wide somatic single-nucleotide variants (SNVs), the method comprising:
identifying double-stranded mutations in a genomic sequence derived from a biological sample, wherein the double-stranded mutations comprise a variant nucleotide on forward and reverse strands; and
performing linkage analysis to obtain a frequency of observed double-stranded mutations adjusted for the number of regions linked to a germline heterozygous variant, thereby measuring the rate of accumulation of genome-wide somatic SNVs.

15. The method of claim 14, wherein the biological sample is a single cell or single nucleus.

16. The method of claim 15, wherein the cell is a neuron, cardiac cell, muscle cell, or skin cell.

17. A method of measuring the somatic mutation burden of a subject, the method comprising:
identifying double-stranded mutations in a genomic sequence from a biological sample from the subject, wherein the double-stranded mutations comprise a variant nucleotide on forward and reverse strands; and
performing linkage analysis to obtain a frequency of observed double-stranded mutations adjusted for the number of regions linked to a germline heterozygous variant, thereby measuring the somatic mutation burden in the subject.

18. The method of claim 17, wherein increased somatic mutation burden in the subject indicates advanced age and/or increased DNA damage.

19. The method of claim 18, wherein the rate of somatic mutation burden of greater than about 20 SNVs per year in the prefrontal cortex (PFC) and/or greater than about 40 SNVs per year in the hippocampal dentate gyrus (DG) is indicative of neuronal degeneration in the subject.

20. The method of claim 19, wherein the neuronal degeneration is associated with Cockayne syndrome (CS) or Xeroderma pigmentosum (XP).
